(19) United States
(12) Patent Application Publication
(10) Pub. No.: US 2004/0243232 A1
(43) Pub. Date: Dec. 2, 2004

(54) Lens for increased depth of focus

(75) Inventor: J. Stuart Cumming, Laguna Beach, CA (US)

Correspondence Address: ORRICK, HERRINGTON & SUTCLIFFE, LLP, 4 PARK PLAZA, SUITE 1600, IRVINE, CA, 92614-2558, US

(73) Assignee: eyeonics, inc

(21) Appl. No.: 10/800,274

(22) Filed: Mar. 12, 2004

Related U.S. Application Data
(63) Continuation-in-part of application No. 10/242,977, filed on Sep. 13, 2002.

(51) Int. Cl. A61F 2/16

(52) U.S. Cl. 623/6.37

(57) ABSTRACT

An intraocular lens provides substantially increased depth of focus for accurate near and far vision with an optic much thinner than a natural lens, and the lens being rigid vaulted posteriorly and adapted for posterior positioning in the capsular bag. The optic is positioned substantially farther from the cornea than a natural lens, so that a cone of light exiting the optic to impinge upon the retina is much smaller than a cone of light from a natural lens. Typically, the optic may be about 1.0 mm thick and its distance from the cornea 7.0-8.0 mm.

This application is continuation-in-part of application Ser. No. 10/242,977, filed Sep. 13, 2002, the disclosure of which is expressly incorporated herein by reference.

### BACKGROUND AND SUMMARY OF THE INVENTION

A natural human optic typically has a thickness of about 5.0 mm. Light rays entering the cornea and passing to the optic typically travel about 7.0 to 8.0 mm. Light rays pass from the optic in a cone of light with its apex at the retina. The natural lens provides only a limited degree of depth of focus with clear vision over a limited range of distances.

The present invention provides an optic which is only a fraction the thickness of the natural lens. Whereas the natural lens is about 5.0 mm thick, the lens of the invention may typically be 1.0 mm and may range from about 0.5 mm to 1.5 mm. The distance from the cornea to the optic of the invention is about 7.0-8.0 mm, whereas with a natural lens, the light rays travel only about 3.5 mm from cornea to optic. Light rays refracted by and exiting the optic define a cone of light much smaller in cross-sectional area than the natural lens, and therefore impinge on the retina in a smaller area. The much smaller cone provides greatly increased depth of focus in comparison with a natural lens, and thus enables clear vision over a long range of distances. In effect, the invention provides effective accommodation as between near and far vision, and a person is enabled to view accurately over a wide range of distances. The optic is positioned much farther from the cornea than a natural lens, and this increase of distance increases the power of the optic required to focus on the retina and minimizes the movement required for a defined change in power in the eye. The further posterior the optic, the higher the power of the optic and the less movement required for a given power change. The lens according to the invention is rigid, the haptics being rigidly connected to the optic, and the lens is vaulted posteriorly. Thus, the distance between the cornea and the optic is maximized and the distance of travel of light rays between cornea and optic is increased. The lens optic is located close to the nodal point of the eye.

The rigid lens causes the optic to move with the periphery of the capsular bag in response to ciliary muscle changes, particularly for near vision.

### BRIEF DESCRIPTION OF THE DRAWINGS

FIG. 1 is a cross-sectional view of a frontal portion of a human eye with a lens according to the invention disposed therein;

FIG. 2 is a partial sectional view of an eye showing light rays entering the cornea and exiting the optic in a cone of light from a natural lens to the retina;

FIG. 3 is a view similar to that of FIG. 2, showing an optic according to the invention, and light rays exiting the optic in a cone of light of smaller size than with the natural lens of FIG. 2;

FIG. 4 and 5 are sectional views taken respectively at line 4-4 and line 5-5 in FIG. 1, showing a capsular bag and haptic in relation to the ciliary muscle in near and far vision positions of the capsular bag and haptic;

FIG. 6 is a diagrammatic sectional view of the ciliary muscle and capsular bag showing in solid lines their near vision positions, and showing in broken lines their far vision positions;

FIG. 7 is an elevational view of a preferred embodiment of lens and haptic according to the invention;

FIG. 8 is a side elevational view of the lens of FIG. 7;

FIG. 9 is an elevational view of another preferred embodiment of lens according to the invention; and

FIG. 10 is a side elevational view of the lens of FIG. 9

### DESCRIPTION OF THE PREFERRED EMBODIMENTS

The present invention provides substantially increased depth of focus, for effective near and far accurate vision by providing a thin optic which is only a fraction the thickness of a natural lens or a conventional artificial lens optic, and by providing a rigid lens adapted to be positioned posteriorly in the natural capsular bag and located close to the nodal point of the eye.

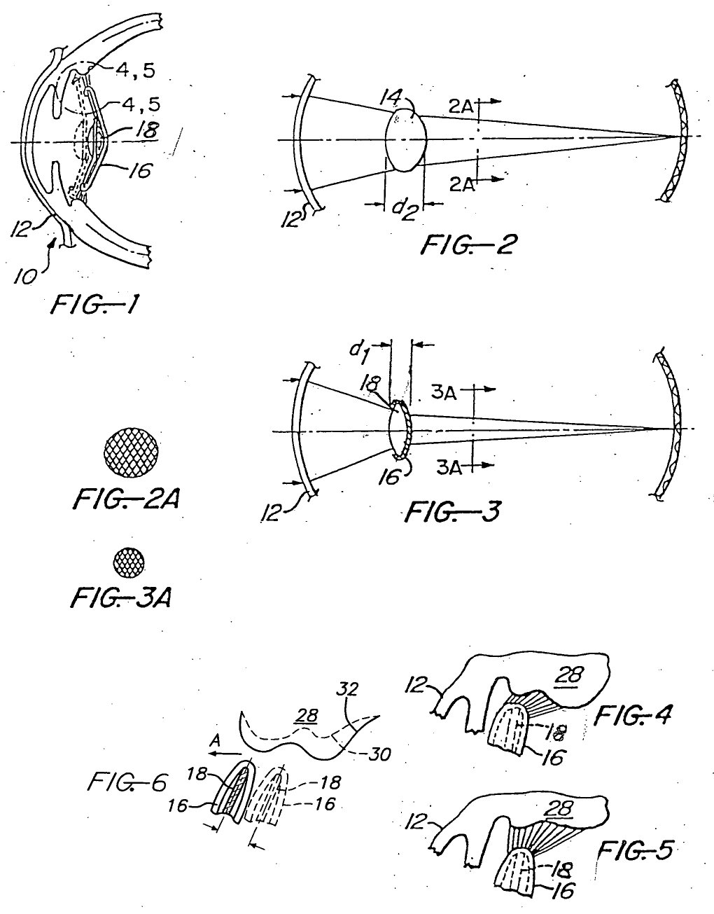

Referring to the drawings, FIG. 1 is a cross-sectional view of an eye 10 with a cornea 12, with a lens 18 according to the invention disposed in the capsular bag 16 of the eye. As indicated in FIG. 2, light rays entering at the cornea are refracted and impact a natural lens 14 which refracts the rays to define a cone of light which impacts the retina. FIG. 3 is a partial sectional view showing a thin optic 18 of the invention disposed substantially farther posteriorly than the natural lens 14 of 5 mm thickness (d2 in FIG. 2), as contracted to a conventional artificial lens of 1.0 mm thickness. The light rays passing from the cornea to the optic 18 must travel a distance of about 7.0 to 8.0 mm from the cornea to the optic, whereas with the natural lens 14 light rays travel only about 3.5 mm. The light rays refracted by and exiting the optic 18 define a cone of light of much smaller cross-sectional area (FIG. 3A) impact the retina in a smaller area, in comparison with the much larger cone of light and its much larger cross section of the human lens (FIGS. 2 and 2A). An optic 18 according to the invention may typically be 1.0 mm thick (d1 in FIG. 3), and may range from about 0.5 to about 1.5 mm in thickness.

The much smaller cone of light provides greatly increased depth of focus, thus enabling clear vision over a long range of distances, in comparison with the much larger cone of light produced by the natural human lens or conventional artificial intraocular lens. The much improved depth of focus provides effective accommodation or “pseudo accommodation”, as between near and far vision, so that a person is enabled to view accurately over a wide range of distances. The increase of distance which light rays must travel between the cornea and the optic minimizes the distance optical power change—i.e., the further posterior the optic, the higher the power of the optic and the less movement required for significant power change.

The lens 18 according to the invention is rigid, with the haptics thereof rigidly connected with the optic. The lens is vaulted posteriorly, as shown in FIGS. 1 and 8, in order to maximize the posterior positioning of the optic to increase the distance of travel of light rays between the cornea and the optic. Additional rigidity may be provided by rigid bars 20 secured along the edges of the lens (FIG. 7), or as shown in FIG. 9 a lens 22 may have rigid bars 24 disposed inwardly of the lens edges with arcuate portions extending about the optic, as shown. The optic is solid but preferably sufficiently flexible to enable folding longitudinally for insertion of the lens into the human eye via a slot therein of relatively short length. Lenses according to the invention may preferably embody upper and lower flexible loop portions 26, 26 (FIG. 7) which extend oppositely to facilitate lens rotation and centration during insertion into an eye, without interfering engagement with the capsular bag. The loop portions 26 preferably are of the same material as the bars 20, but much thinner to be flexible and not rigid like the side bars 20.

The outer peripheral equator portion of the capsular bag is moved in response to configurational changes in the ciliary muscle as between near and far vision, thereby causing the lens and its optic to move with the periphery of the capsular bag in response to such muscle changes, particularly with respect to near vision. That is, upon contraction of the ciliary muscle, anterior displacement of the capsular bag equator effects corresponding anterior movement of the optic. The lens and optic are free to move anteriorly because of the relative stiffness of the anterior bag resulting from leather-like fibrosis or dead tissue arising from conventional surgical techniques. The lens is moved anteriorly and posteriorly only when the muscle acts thereon.

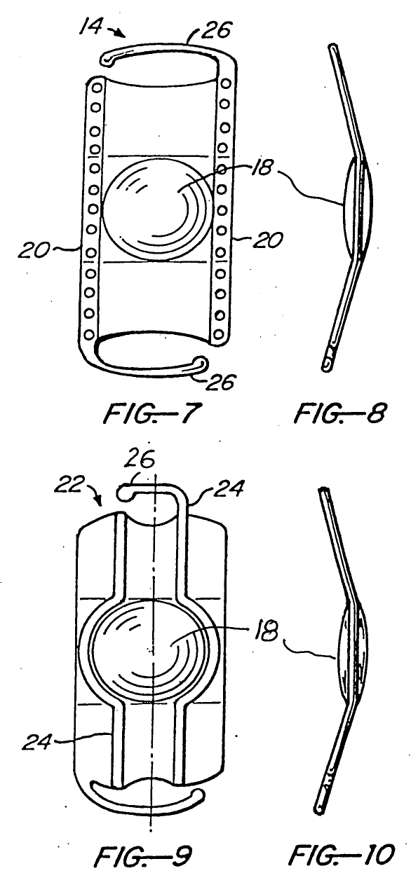

FIGS. 4, 5 and 6 are diagrammatic cross-sectional views of the ciliary muscle 28 of the eye in relation to the peripheral or equator portion of the capsular bag with the lens 18 of the invention therein. FIG. 6 shows in broken lines the configuration 30 of the muscle 28 and the relative position of the lens 18, in a far vision position, and showing in solid lines 32, the muscle configuration and relative position of the lens for near vision. The muscle configuration indicated at 32 extends into the vitreous cavity, thus increasing pressure to a limited degree to further aid in moving the lens anteriorly. Muscle constriction moves the rigid lens 18 forward and backward to a limited degree at the bag periphery.

Thus there has been shown and described a lens for increased depth of focus which fulfills all the objects and advantages sought therefor. Many changes, modifications, variations and other uses and applications of the subject invention will, however, become apparent to those skilled in the art after considering this specification together with the accompanying drawings and claims. All such changes, modifications, variations and other uses and applications which do not depart from the spirit and scope of the invention are deemed to be covered by the invention which is limited only by the claims which follow.

What is claimed is:

1. An intraocular lens for increased depth of focus, comprising:
single solid flexible optic having a thickness substantially less than a natural human lens, and at least two solid rigid haptics connected with the flexible optic, said lens being longitudinally flexible for bending for insertion into an eye and is adapted to be posteriorly positioned in the capsular bag of the eye, whereby light refracted by the cornea travels substantially farther to the optic than with a natural optic and a substantially smaller cone of light passes from the optic to the retina to provide substantially increased depth of focus.

2. A lens according to claim 1, wherein the optic is about 1.0 mm in thickness.

3. A lens according to claim 1, wherein the lens is posterior vaulted and the optic has a thickness between 0.5 mm and 1.5 mm.

4. A lens according to claim 1, wherein the haptics are rigidly connected with the optic and extended therefrom.

5. A lens according to claim 1, wherein the lens is configurated to vault posteriorly in the capsular bag of the eye.

6. A lens according to claim 4, wherein the lens is configurated to vault posteriorly in the capsular bag.

7. A lens according to claim 4, wherein the optic has a thickness between 0.50 mm and 1.5 mm.

8. A lens according to claim 5, wherein the optic has a thickness between 0.60 mm and 1.5 mm.

9. A lens according to claim 4, wherein:
the rigid lens is configured to move anteriorly for near vision and posteriorly for far vision by changes during contraction and relaxation of the ciliary muscle.

10. A lens according to claim 9, wherein:
the rigid lens is adapted to be disposed within the capsular bag and is configured to move about 1.0 mm between their far and near vision positions, whereby the optic is positioned about 1.0 mm further anteriorly than posteriorly to provide improved near vision.

11. An intraocular lens for increased depth of focus, comprising:
a single solid flexible optic having a thickness substantially less than the thickness of a natural human lens, and two solid and rigid haptics rigidly connected to the optic and extending therefrom;
said lens being longitudinally flexible through the optic for bending for insertion into an eye and configurated to vault posteriorly in the capsular bag to position the optic farther from the cornea of the eye,
whereby light refracted by the cornea travels substantially farther to the optic than with a natural optic and a substantially smaller cone of light passes from the optic to the retina to provide substantially increased depth of focus.

12. A posteriorly vaulted and rigid lens according to claim 11, wherein the optic has a thickness between 0.5 mm and 1.5 mm.

13. A posteriorly vaulted lens according to claim 11, wherein:
the lens is adapted to be moved anteriorly for near vision and posteriorly for far vision by changes in pressure within the eye.

14. A posterior vaulted lens according to claim 12, wherein:
the lens is moved anteriorly for near vision and posteriorly for far vision by changes in pressure within the eye.

15. A lens according to claim 13 wherein:
the intraocular lens is configured such that redistribution of ciliary muscle mass upon constriction of the muscle for near vision causes encroachment thereof on the vitreous cavity and an increase of pressure therein to aid in urging the rigid lens anteriorly to enhance near vision.

16. A lens according to claim 13, wherein:
the lens is configured such that the peripheral equator of the capsular bag and the rigid lens therein are adapted to move about 1.0 mm between their far and near vision position, whereby the optic is positioned about 1.0 mm further anteriorly than posteriorly to provide improved near vision.

17. A lens according to claim 11, wherein:
a peripheral equator of the capsular bag and the rigid lens therein are moved forward between their far and near vision positions, whereby the optic is positioned further anteriorly than posteriorly to provide improved near vision.

18. A lens according to claim 4, and further comprising at least one rigid bar secured to and extending longitudinally of the lens to provide rigidity.

19. A lens according to claim 11, and further comprising at least one rigid bar secured to and extending longitudinally of the lens to provide rigidity.

20. A lens according to claim 19, wherein two rigid bars are disposed in spaced relation and extend longitudinally of the lens.
